(19) United States
(12) Patent Application Publication
(10) Pub. No.: US 2024/0419986 A1
(43) Pub. Date: Dec. 19, 2024

(54) METHOD AND APPARATUS FOR CONSTRUCTING KNOWLEDGE BASE, AND METHOD AND APPARATUS FOR GENERATING ANSWER STATEMENT

(71) Applicant: Beijing Jingdong Tuoxian Technology Co., Ltd., Beijing (CN)

(72) Inventors: Jiong Han, Beijing (CN); Yong Wang, Beijing (CN)

(73) Assignee: Beijing Jingdong Tuoxian Technology Co., Ltd., Beijing (CN)

(21) Appl. No.: 18/694,982

(22) PCT Filed: Sep. 6, 2022
(86) PCT No.: PCT/CN2022/117228

§ 371 Date: Mar. 24, 2024

(30) Foreign Application Priority Data
Sep. 26, 2021 (CN) 202111127364.2

(51) Int. Cl. G06N 5/022

(52) U.S. Cl. CPC G06N 5/022

(57) ABSTRACT

A method and an apparatus for constructing a knowledge base are provided. The method includes: acquiring historical inquiry sentences of a plurality of users; performing event extraction on the historic inquiry sentences, to obtain a plurality of extracted events; and constructing, based on the extracted events and corresponding response sentences, a question and answer knowledge base for generating a response sentence during an inquiry of a user [symbol] One or

[symbol]

The disclosure claims the priority of Chinese Patent Application No. 202111127364.2 filed on Sep. 26, 2021, titled “METHOD AND APPARATUS FOR CONSTRUCTING KNOWLEDGE BASE, AND METHOD AND APPARATUS FOR GENERATING RESPONSE SENTENCE”, the entity of which is incorporated herein by reference.

### TECHNICAL FIELD

The present disclosure relates to the field of computer technology, in particular to the field of artificial intelligence technology, and more particularly, to a method and apparatus for constructing a knowledge base, and a method and apparatus for generating a response sentence.

### BACKGROUND

For healthcare apps, it occasionally happens that a doctor butler is unable to respond to messages timely, but patients generally hope to get quick feedbacks on their questions.

Many of the current automated responses rely on keywords for information matching, so it has the following drawbacks: 1. the words in messages sent by patients are not specialized enough and may not involve many professional terms to hit the lexicon, and accurate responses can not be provided; 2. automated answering systems currently on the market do not have a complete knowledge base of recorded doctor-patient interactions, and even if a question is answered, more than one answer may be provided, rather than one accurate answer.

### SUMMARY

Embodiments of the present disclosure provide a method and an apparatus for constructing a knowledge base, a device, and a storage medium.

In one or more embodiments of the present disclosure, an embodiment of the present disclosure provides a method for constructing a knowledge base, the method including: acquiring historical inquiry sentences of a plurality of users; performing event extraction on the historical inquiry sentences, to obtain a plurality of extracted events; and constructing, based on the extracted events and corresponding response sentences, a question and answer knowledge base for generating a response sentence during an inquiry of a user.

In one or more embodiments of the present disclosure, an embodiment of the present disclosure provides a method for generating a response sentence, which includes: acquiring a target inquiry sentence of a target user within a preset historical time period; performing event extraction on the target inquiry sentence to obtain a target extracted event; searching, based on the target extracted event, the question and answer knowledge base for a target response sentence corresponding to the target inquiry sentence, where the question and answer knowledge base is a question and answer knowledge base obtained using the method according to any embodiment; and pushing, in response to determining that the target response sentence is found, the target response sentence to the user.

In one or more embodiments of the present disclosure, an embodiment of the present disclosure provides an apparatus for constructing a knowledge base, which includes: an acquisition module, configured to acquire historical inquiry sentences of a plurality of users; an extraction module, configured to perform event extraction on the historical inquiry sentences, to obtain a plurality of extracted events; and a construction module, configured to construct, based on the extracted events and corresponding response sentences, a question and answer knowledge base for generating a response sentence during an inquiry of a user.

In one or more embodiments of the present disclosure, an embodiment of the present disclosure provides an apparatus for generating an response sentence, which includes: an inquiry module, configured to acquire a target inquiry sentence of a target user within a preset historical time period; an obtaining module, configured to perform event extraction on the target inquiry sentence to obtain a target extracted event; a searching module, configured to search, based on the target extracted event, the question and answer knowledge base for a target response sentence corresponding to the target inquiry sentence, where the question and answer knowledge base is a question and answer knowledge base obtained using the method according to any embodiment; and a pushing module, configured to push, in response to determining that the target response sentence is found, the target response sentence to the user.

In one or more embodiments of the present disclosure, an embodiment of the present disclosure provides an electronic device including at least one processor; and a memory communicatively connected to the at least one processor; where the memory stores instructions executable by the at least one processor, and the instructions, when executed by the at least one processor, cause the at least one processor to perform the method according to any embodiment.

In one or more embodiments of the present disclosure, an embodiment of the present disclosure provides a non-transitory computer readable storage medium storing computer instructions, where the computer instructions are used to cause the computer to perform the method according to any embodiment.

It should be understood that the content described in this section is not intended to identify key or important features of the embodiments of the present disclosure, nor to limit the scope of the disclosure. The other features of the disclosure will be easily understood through the following description.

### BRIEF DESCRIPTION OF THE DRAWINGS

FIG. 1 is an example system architecture diagram to which the present disclosure may be applied;

FIG. 2 is a flowchart of an embodiment of a method for constructing a knowledge base according to the present disclosure:

FIG. 3 is a schematic diagram of an application scenario of the method for constructing a knowledge base according to the present disclosure;

FIG. 4 is a flowchart of another embodiment of the method for constructing a knowledge base according to the present disclosure;

FIG. 5 is a schematic diagram of an embodiment of a method for generating an response sentence according to the present disclosure;

FIG. 6 is a schematic diagram of an embodiment of an apparatus for constructing a knowledge base according to the present disclosure;

FIG. 7 is a schematic diagram of an embodiment of an apparatus for generating an response sentence according to the present disclosure; and

FIG. 8 is a schematic structural diagram of a computer system adapted for implementing a server of embodiments of the present disclosure.

### DETAILED DESCRIPTION OF EMBODIMENTS

Example embodiments of the present disclosure are described below with reference to the accompanying drawings, where various details of the embodiments of the present disclosure are included to facilitate understanding, and should be considered merely as examples. Therefore, those of ordinary skills in the art should realize that various changes and modifications can be made to the embodiments described herein without departing from the scope and spirit of the present disclosure. Similarly, for clearness and conciseness, descriptions of well-known functions and structures are omitted in the following description.

It should be noted that the embodiments and features in the embodiments in the present disclosure may be combined with each other on a non-conflict basis. The present disclosure will be described in detail below with reference to the accompanying drawings and in connection with the embodiments.

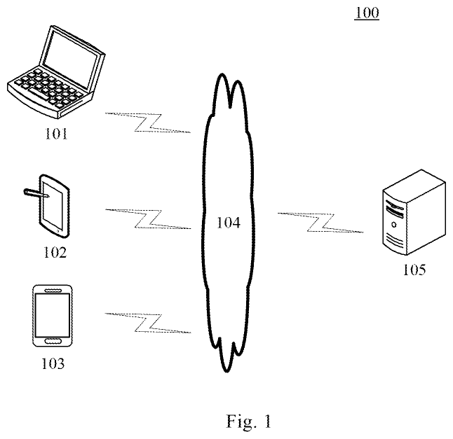

FIG. 1 illustrates an example system architecture 100 to which embodiments of a method for constructing a knowledge base of the present disclosure may be applied.

As shown in FIG. 1, the system architecture 100 may include terminal devices 101, 102, 103, a network 104, and a server 105. The network 104 serves as a medium providing a communication link between the terminal devices 101, 102, 103, and the server 105. The network 104 may include various types of connections, such as wired, wireless communication links or optical fibers.

The terminal devices 101, 102, 103 interact with the server 105 via the network 104 to receive or send messages and the like. Various communication client applications may be installed on the terminal devices 101, 102, 103, such as diagnostic applications, or communication applications.

The terminal devices 101, 102, 103 may be hardware or software. When the terminal devices 101, 102, 103 are hardware, they may be various electronic devices having a display, including but not limited to cell phones and laptop computers. When the terminal devices 101, 102, 103 are software, they may be installed in the electronic devices listed above. They may be implemented as a plurality of software pieces or software modules (e.g., for providing a knowledge base construction service), or may be implemented as a single software piece or software module, which is not specifically limited herein.

The server 105 may be a server that provides various services, such as acquiring historical inquiry sentences of a plurality of users; performing event extraction on the historical inquiry sentences, to obtain a plurality of extracted events; and constructing based on the extracted events and corresponding response sentences a question and answer knowledge base for generating a response sentence during an inquiry of a user.

It should be noted that the server 105 may be hardware or software. When the server 105 is hardware, it may be implemented as a distributed server cluster consisting of multiple servers, or as a single server. When the server is software, it may be implemented as a plurality of software pieces or software modules (e.g., for providing service of constructing a knowledge base), or as a single software piece or software module, which is not limited herein.

It should be noted that the method for constructing a knowledge base provided in embodiments of the present disclosure may be performed by the server 105, or may be performed by the terminal devices 101, 102, 103, or may be performed by the server 105 and the terminal devices 101, 102, 103 in cooperation with each other. Accordingly, various portions (e.g., various units, sub-units, modules, sub-modules) included in the apparatus for constructing a knowledge base may all be arranged in the server 105, or may all be arranged in the terminal devices 101, 102, 103, or may be arranged in the server 105 and the terminal devices 101, 102, 103 respectively.

It should be understood that the numbers of terminal devices, networks, and servers in FIG. 1 are only illustrative. Depending on implementation needs, there may be any number of terminal devices, networks, and servers.

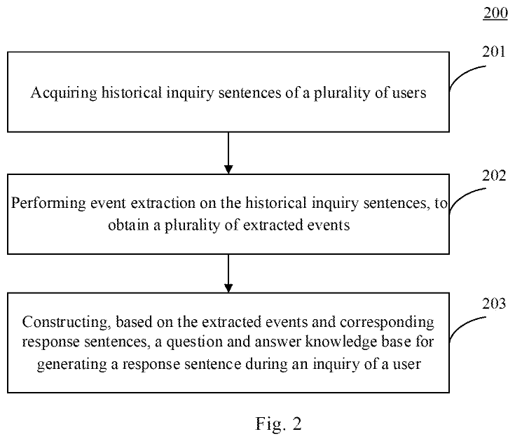

FIG. 2 illustrates a schematic flowchart 200 of a method for constructing a knowledge base that may be applied to the present disclosure. In the present embodiment, the method for constructing a knowledge base includes the following steps 201-203.

Step 201 includes acquiring historical inquiry sentences of a plurality of users.

In the present embodiment, an executing body (such as the server 105 or the terminal devices 101, 102, 103 shown in FIG. 1) may acquire the historical inquiry sentences of the plurality of users from multiple sources, for example, in records of chats between users and doctors stored in a backend database of a health application, in popular science articles of official accounts or periodicals, in chat records of a community forum, etc., which is not limited herein.

Step 202 includes performing event extraction on the historical inquiry sentences, to obtain a plurality of extracted events.

In the present embodiment, after acquiring the historical inquiry sentences of the plurality of users, the executing body may perform event extraction on the historical inquiry sentences using an event extraction algorithm, to obtain the plurality of extracted events.

Event extraction refers to extracting events of interest to a user from unstructured information and presenting the events to the user in a structured way. The event extraction algorithm may use event extraction algorithms in the existing technology or future developing technologies, for example, a pattern matching-based event extraction algorithm, a machine learning-based event extraction algorithm, a neural network-based event extraction algorithm, and so forth, which is not limited herein.

Here, the executing body may evaluate effectiveness of the event extraction algorithm using the following approach:
a micro-averaging value (denoted as F) method based on a recall rate (denoted as R) and accuracy (denoted as P), or a false identification cost (denoted as C) method based on a loss rate (denoted as L) and a false alarm rate (denoted as M), where,

$$F = 2 \times PR ( P + R )$$

$$C = Cmiss \times L \times Ltar + Cfa \times M \times ( 1 - Ltar )$$

Cmiss is the cost of one loss, Cfa is the cost of one false alarm, and Ltar is the a priori probability that the system makes a positive judgment, which is usually set to a constant value according to the specific application. The above formulas show that there is no simple inverse relationship between two effectiveness evaluation approaches, so appropriate conversions should be made when analyzing the effectiveness of two different algorithms using different evaluation approaches.

Step 203 includes constructing, based on the extracted events and corresponding response sentences, a question and answer knowledge base for generating a response sentence during an inquiry of a user.

In the present embodiment, the executing body may construct, based on the acquired plurality of extracted events and the response sentences corresponding to the extracted events in the plurality of extracted events, the question and answer knowledge base for generating a response sentence during an inquiry of a user.

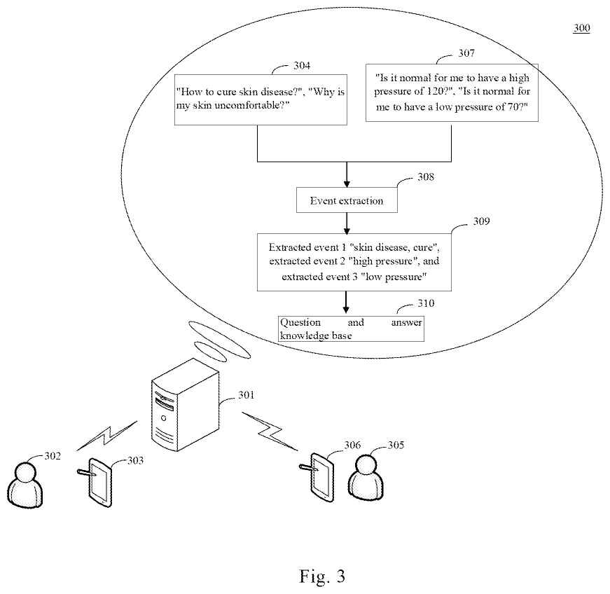

With further reference to FIG. 3, FIG. 3 is a schematic diagram of an application scenario of the method for constructing a knowledge base according to the present embodiment.

In the application scenario of FIG. 3, an executing body 301 acquires historical inquiry sentences of a plurality of users, for example, the historical inquiry sentences 304 entered by user 302 via a terminal device 303 are “How to cure skin disease?” and “Why is skin uncomfortable?”; the historical inquiry sentences 307 entered by user 305 via a terminal device 306 are “Is it normal for me to have a high pressure of 120?” and “Is it normal for me to have a low pressure of 70?”. Further, the executing body performs event extraction 308 on the above historical inquiry sentences, for example, performing event extraction on the historical inquiry sentences of user 1 to obtain extracted events 309, which are extracted event 1 “skin disease, cure”, extracted event 2 “high pressure”, and extracted event 3 “low pressure”, respectively. Response sentences corresponding to the extracted events may be acquired, and based on the extracted events and the corresponding response sentences, a question and answer knowledge base 310 may be constructed, for generating a response sentence during an inquiry of a user.

According to the method for constructing a knowledge base of the present disclosure, historical inquiry sentences of a plurality of users are acquired; event extraction is performed on the historical inquiry sentences, to obtain a plurality of extracted events; and based on the extracted events and corresponding response sentences, a question and answer knowledge base is constructed for generating response sentences during an inquiry of the user, that is, an efficiency and accuracy of generating automated response sentences is improved by constructing a knowledge base applied to user inquiry scenarios.

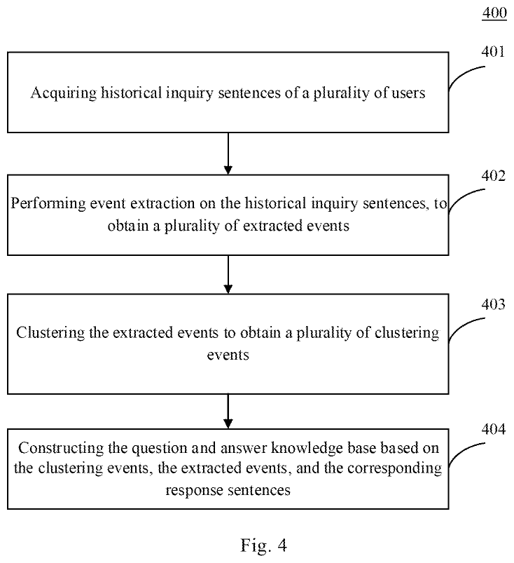

With further reference to FIG. 4, a flow 400 of another embodiment of the method for constructing a knowledge base is illustrated. The flow 400 of the method for constructing a knowledge base in the present embodiment may include the following steps 401-404.

Step 401 includes acquiring historical inquiry sentences of a plurality of users.

In the present embodiment, implementation details and technical effects of step 401 may be referred to the description of step 201, which is not described herein.

Step 402 includes performing event extraction on the historical inquiry sentences, to obtain a plurality of extracted events.

In the present embodiment, implementation details and technical effects of step 402 may be referred to the description of step 202, which is not described herein.

Step 403 includes clustering the extracted events to obtain a plurality of clustering events.

In the present embodiment, the executing body may cluster the plurality of extracted events to obtain the plurality of clustering events using a clustering algorithm.

Here, the clustering algorithm may use clustering algorithms in the existing technology or future developing technologies, for example, the K-means algorithm, the Single-Pass incremental clustering algorithm, the HAC (Hierarchical Agglomerative Clustering) algorithm, and so forth, which is not limited herein.

In particular, for a set of extracted events S={S1, S2, S3, . . . , Sn}, the clustering process is as follows.

(1) a set of categories is initialized for extracted events E={E1, E2, E3, . . . , En}, where Ei=Si.

(2) If for any category Ek and E1, Sim (Ek, E1) is always smaller than Thread, the clustering process is stopped; otherwise, step (3) is proceeded. Sim (Ek, E1) is an inter-category similarity between Ek and E1, which is calculated by the following formula (cosine similarity formula); and Thread is a threshold to specify an end condition of clustering. In embodiments of the disclosure, the clustering is mainly performed for extracted events, so taking 0.2 as the inter-category similarity threshold Thread is reasonable.

$$Sim ( Ei , Ej ) = \frac{{\sum }_{Sk ∈ Ei , Sm ∈ Ej} Sim ( Sk , Sm )}{\vert Ei \vert \vert Ej \vert}$$

(3) Two categories Ei, Ej having the highest inter-category similarity Sim (Ei, Ej) are find and merged, the set E is updated, and step (2) is proceeded.

Step 404 includes constructing the question and answer knowledge base based on the clustering events, the extracted events, and the corresponding response sentences.

In the present embodiment, the executing body may construct, based on the acquired plurality of clustering events corresponding to the plurality of extracted events, the plurality of extracted events, and the response sentences corresponding to the extracted events in the plurality of extracted events, the question and answer knowledge base for generating the response sentences during the inquiry of the user.

In particular, the plurality of extracted events are extracted event 1 “How to cure skin disease?”, and extracted event 2 “How to cure stomach illness?”. The execution body clusters extracted event 1 to obtain clustering event 1 “skin disease inquiry”, and clusters extracted event 2 to obtain clustering event 2 “stomach illness inquiry”. Further, the executing body may construct the question and answer knowledge base, based on clustering event 1, clustering event 2, extracted event 1 and the corresponding response sentence, and extracted event 2 and the corresponding response sentence.

In the above embodiment of the present disclosure, compared with the embodiment corresponding to FIG. 2, the flow 400 of the method for constructing a knowledge base in the present embodiment reflects clustering the extracted events to obtain the plurality of clustering events; and constructing, based on the clustering events, the extracted events, and the corresponding response sentences, the question and answer knowledge base for generating the response sentence during the inquiry of the user, which helps to provide users with more customized treatment plans, improves correlation between question-answer pairs in the question and answer knowledge base, and further improves the efficiency of finding response sentences using the question and answer knowledge base.

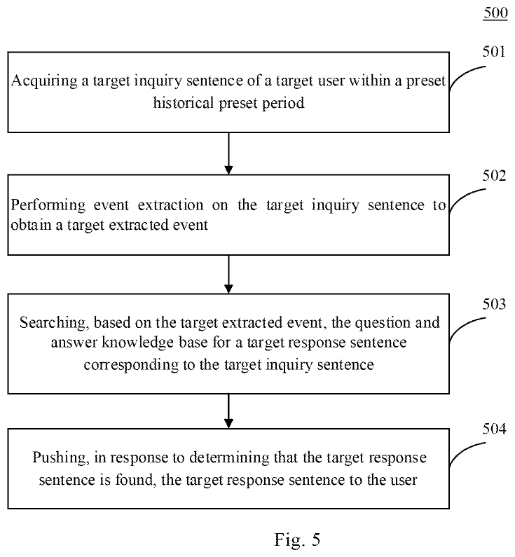

With further reference to FIG. 5, a flow 500 of an embodiment of a method for generating an response sentence illustrated according to the present disclosure. The method for generating an response sentence includes the following steps 501-504.

Step 501 includes acquiring a target inquiry sentence of a target user within a preset historical time period.

In the present embodiment, the executing body may acquire the target inquiry sentence of the target user within the preset historical time period using a wired or wireless approach. The wireless connection approach may include, but is not limited to, a 3G/4G connection, a WiFi connection, a Bluetooth connection, a WiMAX connection, a Zigbee connection, a UWB (ultra wideband) connection, and other wireless connection approaches now known or developed in the future.

Here, the target user may be any user who has a need for inquiry. The preset time period may be set based on experience and actual needs, for example, a preset time period when the doctor is not online, such as a 3-minute period starting from the moment that the doctor is offline, or a 2-minute period starting from the moment that the doctor is offline and the inquiring user asks a first inquiry question, which is not limited herein.

Here, the target inquiry sentence may be one sentence or more than one sentence, which is not limited herein.

Step 502 includes performing event extraction on the target inquiry sentence to obtain a target extracted event.

In the present embodiment, after acquiring target inquiry sentences within the preset historical time period, the executing body may first integrate the target inquiry sentences into a document based on time, then perform event extraction on the document to obtain target extracted events.

In particular, the executing body acquires the target inquiry sentences of the target user within the preset historical time period as “Is it normal for me to have a high pressure of 120?” and “Is it normal for me to have a low pressure of 70?” The executing body performs event extraction on the target inquiry sentences to obtain the target extracted events “high pressure” and “low pressure”.

Step 503 includes searching, based on the target extracted event, the question and answer knowledge base for a target response sentence corresponding to the target inquiry sentence.

In the present embodiment, after acquiring the target extracted event, the executing body may directly search the question and answer knowledge base for a response sentence corresponding to an extracted event corresponding to the target extracted event based on the target extracted event, and determine the response sentence as an answer corresponding to the target inquiry sentence; the executing body may alternatively first perform clustering on the target extracted events to obtain a target clustering event, and search the question and answer knowledge base for an answer corresponding to the target extracted events based on the target clustering event. That is, a clustering event corresponding to the target clustering event is first found in the knowledge base, an extracted event corresponding to the target extracted event is further found under the term of the clustering event, and the response sentence corresponding to the extracted event corresponding to the target extracted event is determined as the answer corresponding to the target inquiry sentence.

The question and answer knowledge base is the knowledge base obtained by using the method as described in the corresponding embodiment of FIG. 2 or FIG. 4, which is not described herein.

In particular, the target inquiry sentences are “Is it normal for me to have a high pressure of 120?” and “Is it normal for me to have a low pressure of 70?”, the executing body performs event extraction on the target inquiry sentences to obtain the target extracted events “high pressure” and “low pressure”. The executing body may respectively search the question and answer knowledge base for the response sentences corresponding to “high pressure” and “low pressure” based on the target extracted events, or may first perform clustering on the target extracted events “high pressure” and “low pressure” to obtain the target clustering event “blood pressure standard”, and then, based on the target clustering event, first find the clustering event corresponding to the target clustering event “blood pressure standard” in the question and answer knowledge base, and then, under the term of the clustering event, search for the extracted events “high pressure” and “low pressure” corresponding to the target extracted events, and then determine the response sentences corresponding to “high pressure” and “low pressure”.

Step 504 includes pushing, in response to determining that the target response sentence is found, the target response sentence to the user.

In the present embodiment, if the executing body finds the target response sentence in the question and answer knowledge base, the executing body may push the target response sentence to the user through a wired or wireless approach.

In some alternative implementations, the method further includes:

updating, in response to determining that the target response sentence is not found, the question and answer knowledge base based on the target extracted event.

In this implementation, if the executing body does not find the target response sentence in the question and answer knowledge base, the executing body may update the question and answer knowledge base based on the target extracted event and the corresponding response sentence, to obtain the updated question and answer knowledge base.

The implementation realizes continuous updating of the question and answer knowledge base by updating the question and answer knowledge base based on the target extracted event, in response to determining that the target response sentence is not found, thus improving an accuracy of the generated response sentences.

In some alternative implementations, the method further includes: correcting, in response to detecting a correction instruction on the target response sentence, the target response sentence based on the correction instruction.

In this implementation, after pushing the response sentence to the user, if the executing body detects the correction instruction on the target response sentence, the executing body may correct the target response sentence based on content indicated by the correction instruction, to obtain the corrected target response sentence, and push the corrected target response sentence to the user.

The implementation further improves an accuracy of the pushed response sentence by correcting the target response sentence based on the correction instruction, in response to detecting the correction instruction on the target response sentence.

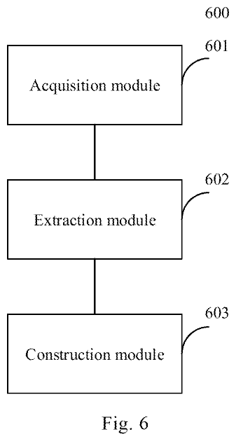

With further reference to FIG. 6, as an implementation of the method shown in the above figures, the present disclosure provides an embodiment of an apparatus for constructing a knowledge base, the embodiment of the apparatus corresponds to the embodiment of the method shown in FIG. 1, and the apparatus may be applied in various electronic devices.

As shown in FIG. 6, the apparatus 600 for constructing a knowledge base in the present embodiment includes: an acquisition module 601, an extraction module 602 and a construction module 603.

Here, the acquisition module 601 may be configured to acquire historical inquiry sentences of a plurality of users.

The extraction module 602 may be configured to perform event extraction on the historical inquiry sentences, to obtain a plurality of extracted events.

The construction module 603 may be configured to construct, based on the extracted events and corresponding response sentences, a question and answer knowledge base for generating response sentences during an inquiry of a user.

In some alternative implementations of the present embodiment, the construction module is further configured to: cluster the extracted events to obtain a plurality of clustering events; and construct the question and answer knowledge base based on the clustering events, the extracted events, and the corresponding response sentences.

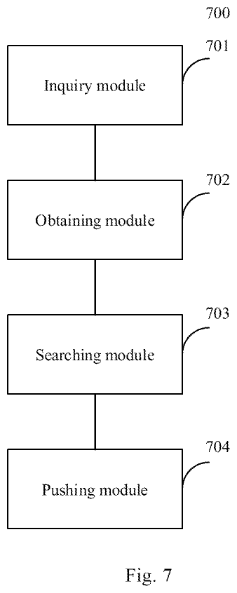

With further reference to FIG. 7, as an implementation of the method shown in the above figures, the present disclosure provides an embodiment of an apparatus for generating a response sentence, the embodiment of the apparatus corresponds to the embodiment of the method shown in FIG. 5, and the apparatus may be applied in various electronic devices.

As shown in FIG. 7, the apparatus 700 for generating a response sentence in the present embodiment includes: an inquiry module 701, an obtaining module 702, a searching module 703 and a pushing module 704.

Here, the inquiry module 701 may be configured to acquire a target inquiry sentence of a target user within a preset historical time period.

The obtaining module 702 may be configured to perform event extraction on the target inquiry sentence to obtain a target extracted event.

The searching module 703 may be configured to search, based on the target extracted event, the question and answer knowledge base for a target response sentence corresponding to the target inquiry sentence.

The pushing module 704 may be configured to push, in response to determining that the target response sentence is found, the target response sentence to the user.

In some alternative implementations of the present embodiment, the apparatus further includes: an updating module, configured to update, in response to determining that the target response sentence is not found, the question and answer knowledge base based on the target extracted event.

In some alternative implementations of the present embodiment, the apparatus further includes: a correction module, configured to correct, in response to detecting a correction instruction on the target response sentence, the target response sentence based on the correction instruction.

According to an embodiment of the present disclosure, the present disclosure also provides an electronic device and a readable storage medium.

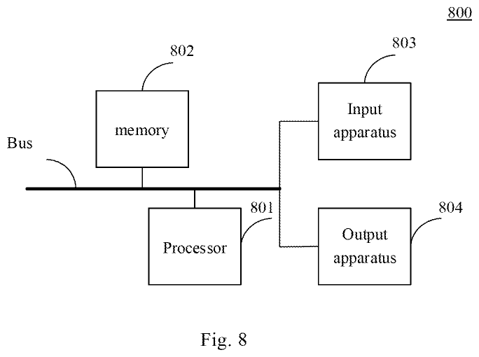

FIG. 8 shows a block diagram of an electronic device of a method for constructing a knowledge base according to embodiments of the present disclosure.

800 is the block diagram of the electronic device of the method for constructing a knowledge base according to embodiments of the present disclosure. The electronic device is intended to represent various forms of digital computers, such as laptop computers, desktop computers, workbenches, personal digital assistants, servers, blade servers, mainframe computers, and other suitable computers. The electronic device may also represent various forms of mobile apparatuses, such as personal digital processors, cellular phones, smart phones, wearable devices, and other similar computing apparatuses. The components shown herein, their connections and relationships, and their functions are merely examples, and are not intended to limit the implementation of the present disclosure described and/or claimed herein.

As shown in FIG. 8, the electronic device includes: one or more processors 801, a memory 802, and interfaces for connecting the various components, including a high-speed interface and a low-speed interface. The components are connected to each other using different buses and may be mounted on a common motherboard or otherwise mounted as desired. The processor may process instructions executed within the electronic device, including instructions stored in or on the memory to display graphical information of the GUI on an external input/output apparatus, such as, a display device coupled to the interface. In other implementations, multiple processors and/or multiple buses may be used with multiple memories and multiple memories, if desired. Similarly, multiple electronic devices may be connected, with the individual devices providing some of the necessary operations (e.g., as an array of servers, a set of blade servers, or a multiprocessor system). One processor 801 is used as an example in FIG. 8.

The memory 802 is a non-transitory computer readable storage medium provided by the present disclosure. The memory stores instructions executable by at least one processor to cause the at least one processor to perform the method for constructing a knowledge base provided in the present disclosure. The non-transitory computer readable storage medium of the present disclosure stores computer instructions, and the computer instructions are used to cause the computer to perform the method for constructing a knowledge base provided in the present disclosure.

The memory 802, as a non-transitory computer readable storage medium, may be used to store non-transitory software programs, non-transitory computer-executable programs, and modules, such as program instructions/modules corresponding to the method for constructing a knowledge base in the embodiments of the present disclosure (e.g., the acquisition module 601, the extraction module 602 and the construction module 603 shown in FIG. 6). The processor 801 executes various functional applications and data processing of the server by running the non-transitory software programs, instructions, and modules stored in the memory 802, i.e., implementing the method for constructing a knowledge base in the above method embodiments.

The memory 802 may include a stored program area and a stored data area, where the stored program area may store an operating system, an application program required by at least one function; and the stored data area may store data created according to the use of the electronic device for updating a cloud platform, etc. Additionally, the memory 802 may include a high-speed random access memory, and may also include a non-transitory memory, such as at least one magnetic disk storage device, a flash memory device, or other non-transitory solid-state storage device. In some embodiments, the memory 802 may optionally include memories located remotely from the processor 801, and these remote memories may be connected to the electronic device for updating a cloud platform via a network. Examples of such network include, but are not limited to, the Internet, an intranet, a local area network, a mobile communication network, and combinations thereof.

The electronic device of the method for constructing a knowledge base may further include: an input apparatus 803 and an output apparatus 804. The processor 801, the memory 802, the input apparatus 803 and the output apparatus 804 may be connected via a bus or in other ways, and connection via a bus is used as an example in FIG. 8.

The input apparatus 803 may receive input digital or character information, such as touch screen, keypad, mouse, trackpad, touchpad, pointing stick, one or more mouse buttons, trackball, joystick and other input apparatuses. The output apparatus 804 may include a display device, an auxiliary lighting apparatus (for example, LED), a tactile feedback apparatus (for example, a vibration motor), and the like. The display device may include, but is not limited to, a liquid crystal display (LCD), a light emitting diode (LED) display, and a plasma display. In some embodiments, the display device may be a touch screen.

Various embodiments of the systems and technologies described herein may be implemented in digital electronic circuit systems, integrated circuit systems, dedicated ASICs (application specific integrated circuits), computer hardware, firmware, software, and/or combinations thereof. These various embodiments may include: being implemented in one or more computer programs that can be executed and/or interpreted on a programmable system that includes at least one programmable processor. The programmable processor may be a dedicated or general-purpose programmable processor, and may receive data and instructions from a storage system, at least one input apparatus, and at least one output apparatus, and transmit the data and instructions to the storage system, the at least one input apparatus, and the at least one output apparatus.

These computing programs (also referred to as programs, software, software applications, or codes) include machine instructions of the programmable processor and may use high-level processes and/or object-oriented programming languages, and/or assembly/machine languages to implement these computing programs. As used herein, the terms “machine readable medium” and “computer readable medium” refer to any computer program product, device, and/or apparatus (for example, magnetic disk, optical disk, memory, programmable logic apparatus (PLD)) used to provide machine instructions and/or data to the programmable processor, including machine readable medium that receives machine instructions as machine readable signals. The term “machine readable signal” refers to any signal used to provide machine instructions and/or data to the programmable processor.

In order to provide interaction with a user, the systems and technologies described herein may be implemented on a computer, the computer has: a display apparatus for displaying information to the user (for example, CRT (cathode ray tube) or LCD (liquid crystal display) monitor); and a keyboard and a pointing apparatus (for example, mouse or trackball), and the user may use the keyboard and the pointing apparatus to provide input to the computer. Other types of apparatuses may also be used to provide interaction with the user; for example, feedback provided to the user may be any form of sensory feedback (for example, visual feedback, auditory feedback, or tactile feedback); and any form (including acoustic input, voice input, or tactile input) may be used to receive input from the user.

The systems and technologies described herein may be implemented in a computing system that includes backend components (e.g., as a data server), or a computing system that includes middleware components (e.g., application server), or a computing system that includes frontend components (for example, a user computer having a graphical user interface or a web browser, through which the user may interact with the implementations of the systems and the technologies described herein), or a computing system that includes any combination of such backend components, middleware components, or frontend components. The components of the system may be interconnected by any form or medium of digital data communication (e.g., communication network). Examples of the communication network include: local area networks (LAN), wide area networks (WAN), the Internet, and blockchain networks.

The computer system may include a client and a server. The client and the server are generally far from each other and usually interact through the communication network. The relationship between the client and the server is generated by computer programs that run on the corresponding computer and have a client-server relationship with each other.

The technical solutions of the embodiments of the present disclosure helps to realize timely intervention in adverse health conditions of users.

It should be understood that the various forms of processes shown above may be used to reorder, add, or delete steps. For example, the steps described in the present disclosure may be performed in parallel, sequentially, or in different orders. As long as the desired results of the technical solution disclosed in the present disclosure can be achieved, no limitation is made herein.

The above specific embodiments do not constitute limitation on the protection scope of the present disclosure. Those skilled in the art should understand that various modifications, combinations, sub-combinations and substitutions may be made according to design requirements and other factors. Any modification, equivalent replacement and improvement made within the spirit and principle of the present disclosure shall be included in the protection scope of the present disclosure.

1. A method for constructing a knowledge base, the method comprising: acquiring historical inquiry sentences of a plurality of users;
performing event extraction on the historical inquiry sentences, to obtain a plurality of extracted events; and
constructing, based on the extracted events and corresponding response sentences, a question and answer knowledge base for generating a response sentence during an inquiry of a user.

2. The method according to claim 1, wherein the constructing, based on the extracted events and corresponding response sentences, a question and answer knowledge base comprises:
clustering the extracted events to obtain a plurality of clustering events; and
constructing the question and answer knowledge base based on the clustering events, the extracted events, and the corresponding response sentences.

3. The method according to claim 1, further comprising:
acquiring a target inquiry sentence of a target user within a preset historical time period;
performing event extraction on the target inquiry sentence to obtain a target extracted event;
searching, based on the target extracted event, the question and answer knowledge base for a target response sentence corresponding to the target inquiry sentence; and
pushing, in response to determining that the target response sentence is found, the target response sentence to the user.

4. The method according to claim 3, wherein the method further comprises:
updating, in response to determining that the target response sentence is not found, the question and answer knowledge base based on the target extracted event.

5. The method according to claim 3, wherein the method further comprises:
correcting, in response to detecting a correction instruction on the target response sentence, the target response sentence based on the correction instruction.

6. An apparatus for constructing a knowledge base, the apparatus comprising:
at least one processor; and
a memory, wherein
the memory stores instructions executable by the at least one processor, and the instructions, when executed by the at least one processor, cause the at least one processor to perform operations comprising:
acquiring historical inquiry sentences of a plurality of users;
performing event extraction on the historical inquiry sentences, to obtain a plurality of extracted events; and
constructing, based on the extracted events and corresponding response sentences, a question and answer knowledge base for generating a response sentence during an inquiry of a user.

7. The apparatus according to claim 6, wherein the operations further comprise:
clustering the extracted events to obtain a plurality of clustering events; and
constructing the question and answer knowledge base based on the clustering events, the extracted events, and the corresponding response sentences.

8. The apparatus according to claim 6, wherein the operations further comprise:
acquiring a target inquiry sentence of a target user within a preset historical time period;
performing event extraction on the target inquiry sentence to obtain a target extracted event;
searching, based on the target extracted event, the question and answer knowledge base for a target response sentence corresponding to the target inquiry sentence; and
pushing, in response to determining that the target response sentence is found, the target response sentence to the user.

9. The apparatus according to claim 8, wherein the operations further comprise:
updating, in response to determining that the target response sentence is not found, the question and answer knowledge base based on the target extracted event.

10. The apparatus according to claim 8, wherein the operations further comprise:
correcting, in response to detecting a correction instruction on the target response sentence, the target response sentence based on the correction instruction.

11. (canceled)

12. A non-transitory computer readable storage medium storing computer instructions, wherein the computer instructions, when executed by a computer, cause the computer to perform operations comprising:
acquiring historical inquiry sentences of a plurality of users;
performing event extraction on the historical inquiry sentences, to obtain a plurality of extracted events; and
constructing, based on the extracted events and corresponding response sentences, a question and answer knowledge base for generating a response sentence during an inquiry of a user.

13. The storage medium according to claim 12, wherein the constructing, based on the extracted events and corresponding response sentences, a question and answer knowledge base comprises:
clustering the extracted events to obtain a plurality of clustering events; and
constructing the question and answer knowledge base based on the clustering events, the extracted events, and the corresponding response sentences.

14. A non-transitory computer readable storage medium storing computer instructions, wherein the computer instructions, when executed by a computer, cause the computer to perform operations comprising:
acquiring historical inquiry sentences of a plurality of users;
performing event extraction on the historical inquiry sentences, to obtain a plurality of extracted events; and
constructing, based on the extracted events and corresponding response sentences, a question and answer knowledge base for generating a response sentence during an inquiry of a user;
acquiring a target inquiry sentence of a target user within a preset historical time period;
performing event extraction on the target inquiry sentence to obtain a target extracted event;
searching, based on the target extracted event, the question and answer knowledge base for a target response sentence corresponding to the target inquiry sentence, wherein the question and answer knowledge base is a question and answer knowledge base obtained according to the method as in claim 1; and
pushing, in response to determining that the target response sentence is found, the target response sentence to the user.

15. The storage medium according to claim 14, wherein the operations further comprise:
updating, in response to determining that the target response sentence is not found, the question and answer knowledge base based on the target extracted event.

16. The storage medium according to claim 14, wherein the operations further comprise:
correcting, in response to detecting a correction instruction on the target response sentence, the target response sentence based on the correction instruction.
